(19) United States
(12) Patent Application Publication
(10) Pub. No.: US 2025/0152752 A1
(43) Pub. Date: May 15, 2025

(54) NEAR-INFRARED FLUORESCENT QUANTUM DOT FOR ACHIEVING RAPID RENAL CLEARANCE, AND PREPARATION METHOD AND APPLICATION THEREOF

(71) Applicant: SUZHOU INSTITUTE OF NANO-TECH AND NANO-BIONICS (SINANO) , CHINESE ACADEMY OF SCIENCES, Suzhou (CN)

(72) Inventors: Qiangbin WANG, Suzhou (CN); Hongchao YANG, Suzhou (CN); Haoying HUANG, Suzhou (CN)

(73) Assignee: SUZHOU INSTITUTE OF NANO-TECH AND NANO-BIONICS (SINANO) , CHINESE ACADEMY OF SCIENCES, Suzhou (CN)

(21) Appl. No.: 18/388,861

(22) Filed: Nov. 13, 2023

(51) Int. Cl. A61K 49/00; A61B 5/00; B82Y 15/00; B82Y 20/00; B82Y 40/00; C09K 11/02; C09K 11/58; C09K 11/60; C09K 11/88

(52) U.S. Cl. CPC A61K 49/0067; A61B 5/0071; A61K 49/0019; C09K 11/025; C09K 11/584; C09K 11/602; C09K 11/881; B82Y 15/00; B82Y 20/00; B82Y 40/00

(57) ABSTRACT

A near-infrared fluorescent quantum dot for achieving rapid renal clearance, and a preparation method and application thereof are provided. The preparation method includes the following steps: carrying out a solvothermal reaction on a first uniformly mixed reaction system including a first probe of a near-infrared fluorescent quantum dot and a weakly polar solvent to prepare a second probe of the near-infrared fluorescent quantum dot with a particle size of less than 5 nm; and then mixing the second probe of near-infrared fluorescent quantum dot with a hydrophilic ligand to form a second uniformly mixed reaction system for a ligand exchange reaction, thus obtaining a near-infrared fluorescent quantum dot for achieving rapid renal clearance, which has a fluorescence emission wavelength ranging from 700 nm to 1700 nm. The near-infrared fluorescent quantum dot for achieving renal clearance is obtained through a simple solvothermal reaction and a subsequent ligand exchange method.

### TECHNICAL FIELD

The present disclosure relates to a near-infrared fluorescent quantum dot and a preparation method thereof, and in particular to a near-infrared fluorescent quantum dot for achieving rapid renal clearance, and a preparation method and application thereof, belonging to the field of material science.

### BACKGROUND

Near-infrared fluorescent quantum dots, as an excellent fluorescent luminescent material, have the following characteristics: high quantum efficiency, tunable excitation and emission wavelength, and easy surface functionalization, etc., and are widely used in the research of in vivo imaging and so on. However, the existing near-infrared fluorescent quantum dots, such as lead sulfide, cadmium telluride, lead selenide, and mercury telluride, include toxic heavy metal elements. Meanwhile, when applied to bioimaging, the residue of quantum dots in organisms also has certain security risks. Therefore, there is an urgent need to develop a novel fluorescent quantum dot material for achieving rapid renal clearance, which has high biocompatibility in a near-infrared window (700-1700 nm).

### SUMMARY

A main objective of the present disclosure is to provide a near-infrared fluorescent quantum dot for achieving rapid renal clearance, and a preparation method thereof, so as to overcome disadvantages in the prior art.

Another objective of the present disclosure is to provide application of the near-infrared fluorescent quantum dot for achieving rapid renal clearance.

To achieve the objectives above, the technical solution adopted by the present disclosure is as follows:

A preparation method of a near-infrared fluorescent quantum dot for achieving rapid renal clearance is provided by embodiments of the present disclosure, including the following steps:
carrying out a solvothermal reaction on a first uniformly mixed reaction system comprising a first probe of a near-infrared fluorescent quantum dot and a weakly polar solvent to prepare a second probe of the near-infrared fluorescent quantum dot with a particle size of less than 5 nm and uniform size; and
carrying out a ligand exchange reaction on a second uniformly mixed reaction system comprising the second probe of near-infrared fluorescent quantum dot and a hydrophilic ligand at 0-100° C. for 1-6000 min, thus obtaining a near-infrared fluorescent quantum dot for achieving rapid renal clearance.

In some embodiments, the preparation method includes: carrying out a solvothermal reaction on the first uniformly mixed reaction system at 0-300° C. for 0.5-24 h, so as to prepare the second probe of near-infrared fluorescent quantum dot.

Further, the first probe of near-infrared fluorescent quantum dot includes any one or a combination of more than two of a selected quantum dot, a Fe-doped selected quantum dot, a Mn-doped selected quantum dot, a Cu-doped selected quantum dot, an Au-doped selected quantum dot, Ag₂S@ZnS, Au-doped Ag₂S@ZnS, Ag₂Te@Ag₂S, Au-doped Ag₂Te@Ag₂S, Ag₂Se@Ag₂S, and Au-doped Ag₂Se@Ag₂S. The selected quantum dot includes any one or a combination of more than two of Ag₂S, Ag₂Se, Ag₂Te, Ag₂SₓSe₁₋ₓ, Ag₂SeₓTe₁₋ₓ, AgAuSe, AgAuTe, AgAuS, AgInS₂, and AgInSe₂, and x is greater than 0 and less than 1.

Further, the hydrophilic ligand includes any one or a combination of more than two of cysteine, cysteamine, glutathione, mercaptopropionic acid, mercapto-1-propanol, mercaptobutyric acid, mercaptobutanol, mercaptoglycerol, mercaptoethylamine, mercaptopropylamine, mercaptoacetic acid, mercaptoethanol, and sulfhydryl glucose.

A near-infrared fluorescent quantum dot for achieving rapid renal clearance prepared by the method above is further provided by the embodiments of the present disclosure.

Further, the near-infrared fluorescent quantum dot for achieving rapid renal clearance has a diameter of less than 5 nm and uniform size distribution.

Further, the near-infrared fluorescent quantum dot for achieving rapid renal clearance has a fluorescence emission peak wavelength ranging from 700 nm to 1700 nm.

Application of any of the near-infrared fluorescent quantum dots for achieving rapid renal clearance above in the field of bioimaging, biomedicine or devices is further provided by the embodiments of the present disclosure, which can achieve rapid renal clearance.

Further, the application includes application of the near-infrared fluorescent quantum dot for achieving rapid renal clearance in a product with a rapid renal clearance function.

Correspondingly, a product with a rapid renal clearance function is further provided by the present disclosure, including a near-infrared fluorescent quantum dot for achieving rapid renal clearance.

Compared with the prior art, the present disclosure has beneficial effects as follows:
(1) A quantum dot is subjected to surface treatment through a simple solvothermal method, and then is subjected to a ligand exchange method to obtain a near-infrared fluorescent quantum dot for achieving rapid renal clearance. A synthesis process of the near-infrared fluorescent quantum dot is simple in step, controllable in experimental conditions, simple and easily available in reagents used, high in yield of final product, and suitable for mass production.
(2) A final product, i.e., a near-infrared fluorescent quantum dot for achieving rapid renal clearance, prepared according to the present disclosure has uniform size distribution, a fluorescent emission tunable from visible light to near infrared and excellent light stability and has no any toxic heavy metal element, and can achieve rapid renal clearance when used in bioimaging, and thus has an important significance.
(3) A preparation process of the present disclosure can also be extended to the preparation processes of other fluorescent quantum dots, and the preparation process is high in yield and easy for the scale-up of reaction.

### BRIEF DESCRIPTION OF THE DRAWINGS

To describe the technical solutions of the present disclosure more clearly, the following briefly introduces the accompanying drawings required for describing the embodiments or the prior art. Apparently, the accompanying drawings in the following description show merely some embodiments of the present disclosure, and those of ordinary skill in the art may still derive other drawings from these accompanying drawings without creative efforts.

FIG. 1 is a transmission electron microscope photo of a near-infrared fluorescent quantum dot for achieving rapid renal clearance according to Embodiment 1 of the present disclosure;

FIG. 2 is a visible-near-infrared absorption spectrogram of a near-infrared fluorescent quantum dot for achieving rapid renal clearance according to Embodiment 1 of the present disclosure;

FIG. 3 is a fluorescence emission pattern of a near-infrared fluorescent quantum dot for achieving rapid renal clearance according to Embodiment 1 of the present disclosure;

FIG. 4A and FIG. 4B are diagrams illustrating a renal clearance process of a near-infrared fluorescent quantum dot for achieving rapid renal clearance according to Embodiment 1 of the present disclosure.

### DETAILED DESCRIPTION OF THE EMBODIMENTS

As above, due to the fact that existing near-infrared fluorescent quantum dots, such as lead sulfide, cadmium telluride, lead selenide, and mercury telluride, include toxic heavy metal element, and when applied to biological imaging, the residue of quantum dots in organisms also has certain security risks, it is of an important significance of designing and synthesizing a near-infrared fluorescent quantum dot for achieving rapid renal clearance. The inventors of this case, through long-term research, have developed a series of quantum dots which do not contain toxic heavy metals. Further, the rapid renal clearance of quantum dots in organisms can be further through surface engineering.

As one aspect of the technical solution of the present disclosure, a preparation method of a near-infrared fluorescent quantum dot for achieving rapid renal clearance is provided, including the following steps:
carrying out a solvothermal reaction on a first uniformly mixed reaction system comprising a first probe of a near-infrared fluorescent quantum dot and a weakly polar solvent to prepare a second probe of the near-infrared fluorescent quantum dot with a particle size less than 5 nm and uniform size; and
carrying out a ligand exchange reaction on a second uniformly mixed reaction system comprising the second probe of near-infrared fluorescent quantum dot and a hydrophilic ligand at 0-100° C. for 1-6000 min, thus obtaining a near-infrared fluorescent quantum dot (which may also be called “a third probe of near-infrared fluorescent quantum dot) for achieving rapid renal clearance.

In some embodiments, the preparation method includes: uniformly mixing a first probe of near-infrared fluorescent quantum dot with a weakly polar solvent to form the first uniformly mixed reaction system.

In some embodiments, a mass ratio of the first probe of near-infrared fluorescent quantum dot to the weakly polar solvent is 1-10:10-1000.

In some embodiments, the preparation method specifically includes: carrying out a solvothermal reaction on the first uniformly mixed reaction system at 0-300° C. for 0.5-24 h, preferably 1-6 h, so as to prepare a second probe of the near-infrared fluorescent quantum dot.

In some embodiments, the preparation method specifically includes: uniformly mixing the second probe of near-infrared fluorescent quantum dot with a hydrophilic ligand to form the second uniformly mixed reaction system.

In some embodiments, a mass ratio of the second probe of near-infrared fluorescent quantum dot to the hydrophilic ligand is 1-10:1-10.

Further, the first probe of near-infrared fluorescent quantum dot includes any one or a combination of more than two of a selected quantum dot, a Fe-doped selected quantum dot, a Mn-doped selected quantum dot, a Cu-doped selected quantum dot, an Au-doped selected quantum dot, Ag₂S@ZnS, Au-doped Ag₂S@ZnS, Ag₂Te@Ag₂S, Au-doped Ag₂Te@Ag₂S, Ag₂Se@Ag₂S, and Au-doped Ag₂Se@Ag₂S.

The selected quantum dot includes any one or a combination of more than two of Ag₂S, Ag₂Se, Ag₂Te, Ag₂SₓSe₁₋ₓ, Ag₂SeₓTe₁₋ₓ, AgAuSe, AgAuTe, AgAuS, AgInS₂, and AgInSe₂, and x is greater than 0 and less than 1.

Specifically, the first probe of the near-infrared fluorescent quantum dot includes, but is not limited to, any one or a combination of more than two of Ag₂S, Ag₂Se, Ag₂Te, Ag₂SₓSe₁₋ₓ, Ag₂SeₓTe₁₋ₓ, AgAuSe, AgAuTe, AgAuS, AgInS₂, AgInSe₂, Fe-doped Ag₂S, Fe-doped Ag₂Se, Fe-doped Ag₂Te, Fe-doped AgInS₂, Fe-doped AgInSe₂, Mn-doped Ag₂S, Mn-doped Ag₂Se, Mn-doped Ag₂Te, Mn-doped Ag₂SₓSe₁₋ₓ, Mn-doped Ag₂SeₓTe₁₋ₓ, Mn-doped AgAuSe, Mn-doped AgAuTe, Mn-doped AgAuS, Mn-doped AgInS₂, Mn-doped AgInSe₂, Cu-doped Ag₂S, Cu-doped Ag₂Se, Cu-doped Ag₂Te, Cu-doped Ag₂SₓSe₁₋ₓ, Cu-doped Ag₂SeₓTe₁₋ₓ, Cu-doped AgAuSe, Cu-doped AgAuTe, Cu-doped AgAuS, Cu-doped AgInS₂, Cu-doped AgInSe₂, Au-doped Ag₂S, Au-doped Ag₂Se, Au-doped Ag₂Te, Au-doped AgInS₂, Au-doped AgInSe₂, Ag₂S@ZnS, Ag₂Te@Ag₂S, and Ag₂Se@Ag₂S, in which x is greater than 0 and less than 1.

In some embodiments, the weakly polar solvent includes, but is not limited to, any one or a combination of more than two of oleylamine, oleic acid, octadecene, octadecylamine, dodecylamine, tetradecylamine, dodecanethiol, octanethiol, hexadecanethiol, and octadecanethiol.

In some embodiments, the hydrophilic ligand includes, but is not limited to, any one or a combination of more than two of cysteine, cysteamine, glutathione, mercaptopropionic acid, mercapto-1-propanol, mercaptobutyric acid, mercaptobutanol, mercaptoglycerol, mercaptoethylamine, mercaptopropylamine, mercaptoacetic acid, mercaptoethanol, and sulfhydryl glucose.

Certainly, the near-infrared fluorescent quantum dot, the weakly polar solvent and the hydrophilic ligand may be selected from, but are not limited to, the types listed above.

Further, in a typical embodiment, the preparation method may include: dispersing the first probe of near-infrared fluorescent quantum dot in octanethiol to react at 0-300° C. for 1-6 h, cleaning, and dispersing in trichloromethane to obtain a second probe of near-infrared fluorescent quantum dot.

Further, a mass ratio of the first probe of near-infrared fluorescent quantum dot to the weakly polar solvent is 0.1-1 g:1-100 g.

In some preferred embodiments, the preparation method may include: dissolving a first probe of the near-infrared fluorescent quantum dot with a mass of 0.1-1 g in the weakly polar solvent, i.e., octanethiol.

Further, the preparation method specifically includes: mixing the second probe of near-infrared fluorescent quantum dot with a hydrophilic ligand to react at 0-100° C., so as to obtain a third probe of near-infrared fluorescent quantum dot for achieving rapid renal clearance.

Further, the preparation method may include: cleaning the third probe of near-infrared fluorescent quantum dot after the reaction is completed.

As one of more specific embodiments, the preparation method may include the following steps:
I, mixing a first probe of near-infrared fluorescent quantum dot with a weakly polar solvent, then uniformly dispersing the mixture by ultrasound, and reacting at 0-300° C. for 0.5-24 h;
II, separating out a product obtained through the reaction in Step I, cleaning, and dispersing the product in trichloromethane; and
III, reacting a product obtained in Step II with a hydrophilic ligand at 0-100° C. for 1-6000 min, so as to obtain the third probe of near-infrared fluorescent quantum dot.

According to the present disclosure, the third probe of near-infrared fluorescent quantum dot for achieving renal clearance is obtained through a simple solvothermal reaction and a subsequent ligand exchange method. A synthesis process of the third probe of near-infrared fluorescent quantum dot is simple and controllable, high in yield and for achieving mass preparation. The obtained product is uniform in size, has a fluorescence emission from visible light to a near-infrared region, and can achieve rapid renal clearance in mice, and thus has a wide application prospect in the field of biological imaging.

As another aspect of the technical solution of the present disclosure, a near-infrared fluorescent quantum dot for achieving rapid renal clearance prepared by the method above is also provided.

Further, the near-infrared fluorescent quantum dot for achieving rapid renal clearance has a diameter of less than 5 nm and uniform size distribution.

Further, the near-infrared fluorescent quantum dot for achieving rapid renal clearance has a fluorescence emission peak wavelength ranging from 700 nm to 1700 nm, preferably from 1000 nm to 1300 nm.

A final product, i.e., the third probe of near-infrared fluorescent quantum dot, prepared by the present disclosure has a uniform size distribution, an emission peak wavelength ranging from 700 nm to 1700 nm, and has no any toxic heavy metal elements. The final product yield is high, and the preparation process is easy for scale-up of the reaction scale. In addition, the probe can achieve rapid renal clearance in organisms, and has an important application prospect in the aspect of biomedical imaging.

Another aspect of the embodiments of the present disclosure also provides application of any of the near-infrared fluorescent quantum dots for achieving rapid renal clearance above in the field of bioimaging, biomedicine or devices, which can achieve rapid renal clearance.

Further, the application includes application of the near-infrared fluorescent quantum dot for achieving rapid renal clearance in a product with a rapid renal clearance function.

Another aspect of the embodiments of the present disclosure also provides a product with a rapid renal clearance function, including above near-infrared fluorescent quantum dot for achieving rapid renal clearance.

In conclusion, by means of the technical solution above, a quantum dot is subjected to surface treatment through a simple solvothermal method, and then is subjected to a ligand exchange method to obtain a near-infrared fluorescent quantum dot for achieving rapid renal clearance. A synthesis process of the near-infrared fluorescent quantum dot is simple in step, controllable in experimental conditions, simple and easily available in reagents used, high in yield of final product, and suitable for mass production.

In order to make the objectives, technical solution and advantages of the present disclosure clearer, the technical solution of the present disclosure will be further described in detail below with reference to several preferred embodiments, but the present disclosure is not limited to the following embodiments, and the non-essential improvements and adjustments made by those skilled in the art under the core guiding ideology of the present disclosure still fall within the scope of protection of the present disclosure. Unless specifically stated, various reagents used in the following embodiments are well known to those skilled in the art and are available on a commercial basis or the like. However, experimental methods without specific conditions specified in the following examples are usually in accordance with conventional conditions, or conditions recommended by manufacturers.

### Embodiment 1

0.06 g of first probe of Ag₂S quantum dot was dissolved in 20 mL of oleylamine, and dispersed uniformly by ultrasound for reaction at 30° C. for 24 h, so as to obtain a second probe of Ag₂S quantum dot. Then, 0.6 g of glutathione was added for reaction at 20° C. for 2 h, so as to obtain a third probe of Ag₂S fluorescent quantum dot for achieving rapid renal clearance.

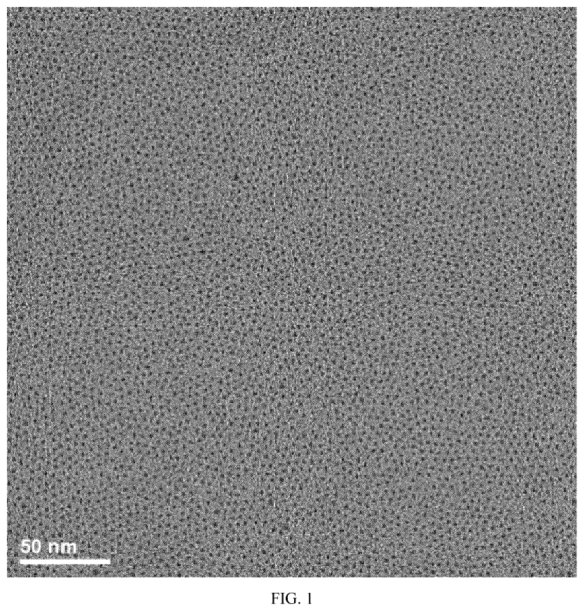

As can be seen from FIG. 1, the third probe of Ag₂S fluorescent quantum dot product for achieving rapid renal clearance obtained according to this embodiment is uniform in morphology and size, and the size is about 3 nm.

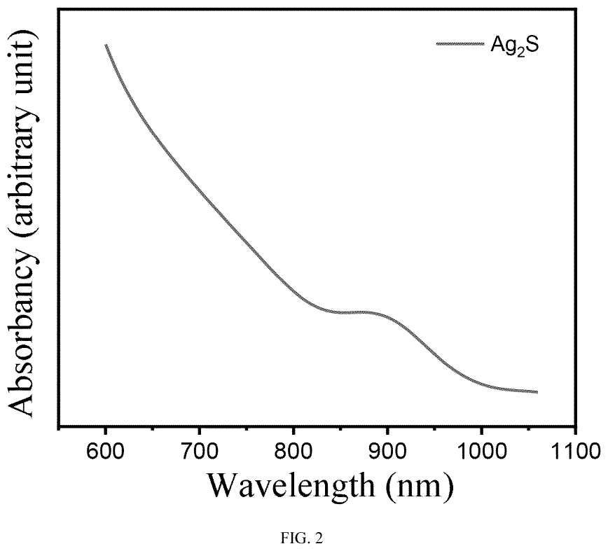

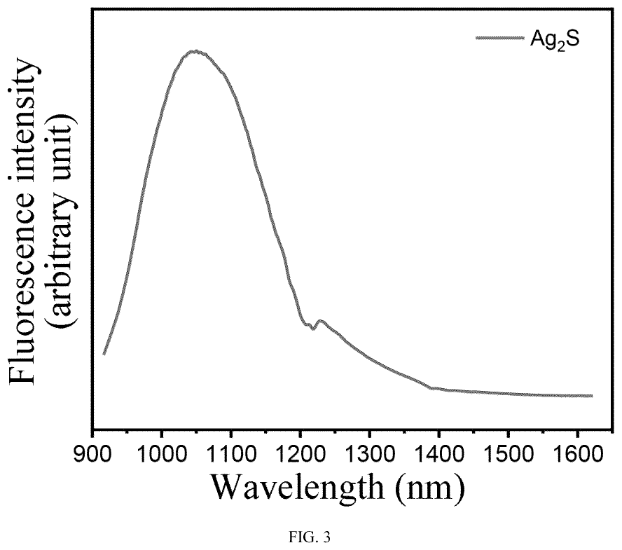

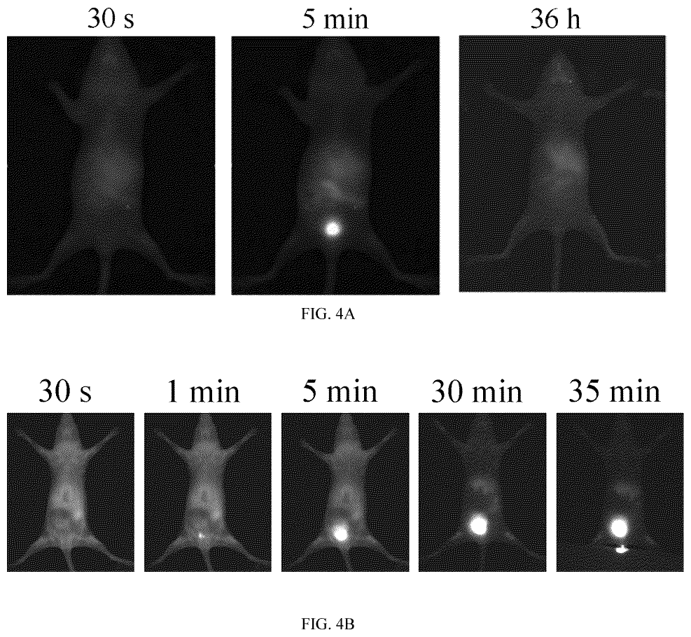

The third probe of Ag₂S quantum dot is dispersed in water. An absorption spectrum of the third probe of Ag₂S quantum dot is measured using a visible-near infrared absorption spectrometer, and it can be seen that the quantum dot has strong absorption from visible to near infrared region, as shown in FIG. 2. A luminescence spectrum of the Ag₂S quantum dot is further measured using a near-infrared fluorescence spectrometer. As can be known from FIG. 3, an emission peak of the third probe of Ag₂S fluorescent quantum dot is located at 1050 nm. As can be seen from FIG. 4A and FIG. 4B, the quantum dot begins to accumulate in the bladder after 5 min of injection, and is completely eliminated from the body after 36 h of circulation, thus achieving renal clearance.

### Embodiment 2

0.06 g of first probe of Ag₂Se quantum dot was dissolved in 20 mL of octanethiol, and dispersed uniformly by ultrasound for reaction at 0° C. for 24 h, so as to obtain a second probe of Ag₂Se quantum dot. Then, 0.06 g of cysteine was added for reaction at 100° C. for 1 min, so as to obtain a third probe of Ag₂Se fluorescent quantum dot for achieving rapid renal clearance.

### Embodiment 3

0.6 g of first probe of Ag₂Te quantum dot was dissolved in 20 mL of hexadecanethiol, and dispersed uniformly by ultrasound for reaction at 200° C. for 0.5 h, so as to obtain a second probe of Ag₂Te quantum dot. Then, 0.6 g of mercapto-1-propanol was added for reaction at 0° C. for 6000 min, so as to obtain a third probe of Ag₂Te fluorescent quantum dot for achieving rapid renal clearance.

### Embodiment 4

0.06 g of first probe of AgAuSe quantum dot was dissolved in 20 mL of dodecylamine, and dispersed uniformly by ultrasound for reaction at 50° C. for 1 h, so as to obtain a second probe of AgAuSe quantum dot. Then, 0.06 g of cysteamine was added for reaction at 30° C. for 600 min, so as to obtain a third probe of AgAuSe fluorescent quantum dot for achieving rapid renal clearance.

### Embodiment 5

0.06 g of first probe of Ag₂SₓSe₁₋ₓ quantum dot was dissolved in 20 mL of octanethiol, and dispersed uniformly by ultrasound for reaction at 100° C. for 2 h, so as to obtain a second probe of Ag₂SₓSe₁₋ₓ quantum dot. Then, 0.6 g of mercaptoglycerol was added for reaction at 20° C. for 1000 min, so as to obtain a third probe of Ag₂SₓSe₁₋ₓ fluorescent quantum dot for achieving rapid renal clearance.

### Embodiment 6

0.6 g of first probe of Ag₂SeₓTe₁₋ₓ quantum dot was dissolved in 20 mL of dodecanethiol, and dispersed uniformly by ultrasound for reaction at 80° C. for 4 h, so as to obtain a second probe of Ag₂SeₓTe₁₋ₓ quantum dot. Then, 0.6 g of mercaptoethanol was added for reaction at 10° C. for 2000 min, so as to obtain a third probe of Ag₂SeₓTe₁₋ₓ fluorescent quantum dot for achieving rapid renal clearance.

### Embodiment 7

0.6 g of first probe of Fe-doped Ag₂S quantum dot was dissolved in 20 mL of dodecanethiol, and dispersed uniformly by ultrasound for reaction at 300° C. for 0.5 h, so as to obtain a second probe of Fe-doped Ag₂S quantum dot. Then, 0.6 g of mercaptoacetic acid was added for reaction at 30° C. for 600 min, so as to obtain a third probe of Fe-doped Ag₂S fluorescent quantum dot for achieving rapid renal clearance.

### Embodiment 8

0.1 g of first probe of Cu-doped Ag₂Se quantum dot was dissolved in 20 mL of dodecanethiol, and dispersed uniformly by ultrasound for reaction at 100° C. for 1 h, so as to obtain a second probe of Cu-doped Ag₂Se quantum dot. Then, 0.6 g of mercaptopropionic acid was added for reaction at 100° C. for 100 min, so as to obtain a third probe of Cu-doped Ag₂Se fluorescent quantum dot for achieving rapid renal clearance.

### Embodiment 9

0.4 g of first probe of Mn-doped Ag₂Te quantum dot was dissolved in 10 mL of dodecanethiol and 10 mL of oleylamine, and dispersed uniformly by ultrasound for reaction at 50° C. for 1 h, so as to obtain a second probe of Mn-doped Ag₂Te quantum dot. Then, 2 g of mercaptoethanol was added for reaction at 10° C. for 2000 min, so as to obtain a third probe of Mn-doped Ag₂Te fluorescent quantum dot for achieving rapid renal clearance.

### Embodiment 10

1 g of first probe of Au-doped AgInSe₂ quantum dot was dissolved in 10 mL of octanethiol and 5 mL of oleic acid, and dispersed uniformly by ultrasound for reaction at 100° C. for 2 h, so as to obtain a second probe of Au-doped AgInSe₂ quantum dot. Then, 0.6 g of mercaptoethanol was added for reaction at 100° C. for 2000 min, so as to obtain a third probe of Au-doped AgInSe, fluorescent quantum dot capable of achieving rapid renal clearance.

### Embodiment 11

0.1 g of first probe of Au-doped Ag₂S@ZnS quantum dot was dissolved in 10 mL of dodecanethiol and 2 mL of octadecene, and dispersed uniformly by ultrasound for reaction at 50° C. for 6 h, so as to obtain a second probe of Au-doped Ag₂S@ZnS quantum dot. Then, 2 g of mercaptoethanol was added for reaction at 30° C. for 600 min, so as to obtain a third probe of Au-doped Ag₂S@ZnS quantum dot capable of achieving rapid renal clearance.

In addition, the inventors of this case also use other raw materials and other process conditions listed above to replace various raw materials and corresponding process conditions in Embodiments 1-11 for corresponding experiments. For example, AgAuTe, AgAuS, AgInS₂ and other quantum dots are used to replace the Ag₂S quantum dot in Embodiment 1. For another example, the Au-doped Ag₂Te@Ag₂S, Au-doped Ag₂Se@Ag₂S and other quantum dots are used to replace the Ag₂S@ZnS quantum dot in Embodiment 1. For still another example, mercaptobutyric acid, mercaptobutanol, mercaptoethylamine, mercaptopropylamine, sulfhydryl glucose or the like is as the hydrophilic ligand to replace the glutathione in Embodiment 1. Therefore, the morphology and performance of the obtained near-infrared fluorescent quantum dot capable of achieving rapid renal clearance are ideal, which are basically similar to the products in Embodiments 1-11.

According to the present disclosure, a second of near-infrared fluorescent quantum dot with particle size less than 5 nm and uniform size is prepared through a simple solvothermal reaction, and is further subjected to a ligand exchange reaction to obtain a third probe of near-infrared fluorescent quantum dot capable of achieving renal clearance. A synthesis process of the third probe of near-infrared fluorescent quantum dot is simple and controllable, high in yield, and capable of achieving large-scale preparation. The obtained product has a uniform size and a fluorescence emission located in the near infrared, and can achieve rapid renal clearance when applied to bioimaging.

Although the present disclosure has been described with reference to illustrative embodiments, those skilled in the art should appreciate that various other changes, omissions and/or additions may be made without departing from the spirit and scope of the present disclosure, and substantial equivalents may be substituted for the elements of the described embodiments. Additionally, many modifications may be made to adapt particular situations or materials to the teachings of the present disclosure without departing from the scope of the present disclosure. Accordingly, it is not intended herein to limit the present disclosure to the particular embodiments disclosed for carrying out the present disclosure, but rather to encompass all embodiments falling within the scope of the appended claims. Furthermore, unless specifically stated, any use of the terms first, second, etc. does not indicate any order or importance, but rather, the use of the terms first, second, etc. is to distinguish one element from another.

What is claimed is:

1. A preparation method of a near-infrared fluorescent quantum dot for achieving a rapid renal clearance, comprising:
carrying out a solvothermal reaction on a first uniformly mixed reaction system comprising a first probe of the near-infrared fluorescent quantum dot and a weakly polar solvent to prepare a second probe of the near-infrared fluorescent quantum dot with a particle size of less than 5 nm and uniform size; and
carrying out a ligand exchange reaction on a second uniformly mixed reaction system comprising the second probe of the near-infrared fluorescent quantum dot and a hydrophilic ligand at 0-100° C. for 1-6000 min to obtain the near-infrared fluorescent quantum dot for achieving the rapid renal clearance.

2. The preparation method according to claim 1, wherein the preparation method comprises: uniformly mixing the first probe of the near-infrared fluorescent quantum dot with the weakly polar solvent to form the first uniformly mixed reaction system, wherein a mass ratio of the first probe of the near-infrared fluorescent quantum dot to the weakly polar solvent is 1-10:10-1000.

3. The preparation method according to claim 1, wherein the preparation method comprises: carrying out the solvothermal reaction on the first uniformly mixed reaction system at 0-300° C. for 0.5-24 h to prepare the second probe of the near-infrared fluorescent quantum dot.

4. The preparation method according to claim 1, wherein the preparation method comprises: uniformly mixing the second probe of the near-infrared fluorescent quantum dot with the hydrophilic ligand to form the second uniformly mixed reaction system, wherein a mass ratio of the second probe of the near-infrared fluorescent quantum dot to the hydrophilic ligand is 1-10:1-10.

5. The preparation method according to claim 1, wherein the first probe of the near-infrared fluorescent quantum dot comprises one or a combination of more than two of a selected quantum dot, a Fe-doped selected quantum dot, a Mn-doped selected quantum dot, a Cu-doped selected quantum dot, an Au-doped selected quantum dot, Ag₂S@ZnS, Au-doped Ag₂S@ZnS, Ag₂Te@Ag₂S, Au-doped Ag₂Te@Ag₂S, Ag₂Se@Ag₂S, and Au-doped Ag₂Se@Ag₂S, and wherein the selected quantum dot comprises one or a combination of more than two of Ag₂S, Ag₂Se, Ag₂Te, Ag₂SₓSe₁₋ₓ, Ag₂SeₓTe₁₋ₓ, AgAuSe, AgAuTe, AgAuS, AgInS₂, and AgInSe₂, and x is greater than 0 and less than 1.

6. The preparation method according to claim 1, wherein the weakly polar solvent comprises one or a combination of more than two of oleylamine, oleic acid, octadecene, octadecylamine, dodecylamine, tetradecylamine, dodecanethiol, octanethiol, hexadecanethiol, and octadecanethiol.

7. The preparation method according to claim 1, wherein the hydrophilic ligand comprises one or a combination of more than two of cysteine, cysteamine, glutathione, mercaptopropionic acid, mercapto-1-propanol, mercaptobutyric acid, mercaptobutanol, mercaptoglycerol, mercaptoethylamine, mercaptopropylamine, mercaptoacetic acid, mercaptoethanol, and sulfhydryl glucose.

8. A near-infrared fluorescent quantum dot for achieving a rapid renal clearance prepared by the preparation method according to claim 1, wherein the near-infrared fluorescent quantum dot for achieving the rapid renal clearance has a diameter of less than 5 nm and a uniform size distribution, and has a fluorescence emission peak wavelength ranging from 700 nm to 1700 nm.

9. A method of an application of the near-infrared fluorescent quantum dot for achieving the rapid renal clearance according to claim 8 in the field of bioimaging, biomedicine or devices, wherein the near-infrared fluorescent quantum dot for achieving rapid renal clearance is configured in preparing a product with a rapid renal clearance function.

10. A product with a rapid renal clearance function, comprising the near-infrared fluorescent quantum dot for achieving the rapid renal clearance according to claim 8.

11. The preparation method according to claim 3, wherein in the preparation method, the solvothermal reaction is carried out on the first uniformly mixed reaction system at 0-300° C. for 1-6 h.

12. The near-infrared fluorescent quantum dot for achieving the rapid renal clearance according to claim 8, wherein the near-infrared fluorescent quantum dot for achieving the rapid renal clearance has the fluorescence emission peak wavelength ranging from 1000 nm to 1300 nm.

13. The near-infrared fluorescent quantum dot for achieving the rapid renal clearance according to claim 8, wherein the preparation method comprises: uniformly mixing the first probe of the near-infrared fluorescent quantum dot with the weakly polar solvent to form the first uniformly mixed reaction system, wherein a mass ratio of the first probe of the near-infrared fluorescent quantum dot to the weakly polar solvent is 1-10:10-1000.

14. The near-infrared fluorescent quantum dot for achieving the rapid renal clearance according to claim 8, wherein the preparation method comprises: carrying out the solvothermal reaction on the first uniformly mixed reaction system at 0-300° C. for 0.5-24 h to prepare the second probe of the near-infrared fluorescent quantum dot.

15. The near-infrared fluorescent quantum dot for achieving the rapid renal clearance according to claim 8, wherein the preparation method comprises: uniformly mixing the second probe of the near-infrared fluorescent quantum dot with the hydrophilic ligand to form the second uniformly mixed reaction system, wherein a mass ratio of the second probe of the near-infrared fluorescent quantum dot to the hydrophilic ligand is 1-10:1-10.

16. The near-infrared fluorescent quantum dot for achieving the rapid renal clearance according to claim 8, wherein in the preparation method, the first probe of the near-infrared fluorescent quantum dot comprises one or a combination of more than two of a selected quantum dot, a Fe-doped selected quantum dot, a Mn-doped selected quantum dot, a Cu-doped selected quantum dot, an Au-doped selected quantum dot, Ag₂S@ZnS, Au-doped Ag₂S@ZnS, Ag₂Te@Ag₂S, Au-doped Ag₂Te@Ag₂S, Ag₂Se@Ag₂S, and Au-doped Ag₂Se@Ag₂S, and wherein the selected quantum dot comprises one or a combination of more than two of Ag₂S, Ag₂Se, Ag₂Te, Ag₂SₓSe₁₋ₓ, Ag₂SeₓTe₁₋ₓ, AgAuSe, AgAuTe, AgAuS, AgInS₂, and AgInSe₂, and x is greater than 0 and less than 1.

17. The near-infrared fluorescent quantum dot for achieving the rapid renal clearance according to claim 8, wherein in the preparation method, the weakly polar solvent comprises one or a combination of more than two of oleylamine, oleic acid, octadecene, octadecylamine, dodecylamine, tetradecylamine, dodecanethiol, octanethiol, hexadecanethiol, and octadecanethiol.

18. The near-infrared fluorescent quantum dot for achieving the rapid renal clearance according to claim 8, wherein in the preparation method, the hydrophilic ligand comprises one or a combination of more than two of cysteine, cysteamine, glutathione, mercaptopropionic acid, mercapto-1-propanol, mercaptobutyric acid, mercaptobutanol, mercaptoglycerol, mercaptoethylamine, mercaptopropylamine, mercaptoacetic acid, mercaptoethanol, and sulfhydryl glucose.
